(19) United States
(12) Patent Application Publication
(10) Pub. No.: US 2013/0345731 A1
(43) Pub. Date: Dec. 26, 2013

(54) Infection control scalpel

(71) Applicants: Raj Singh, Oakland, CA (US); Aaron Chapman, Ashland, OR (US)

(72) Inventors: Raj Singh, Oakland, CA (US); Aaron Chapman, Ashland, OR (US)

(21) Appl. No.: 13/199,937

(22) Filed: Sep. 14, 2011

(51) Int. Cl. A61B 19/00; A61B 17/3211

(52) U.S. Cl. CPC A61B 19/42; A61B 17/3211
USPC 606/167

(57) ABSTRACT

This is a surgical scalpel that has a skirt preventing blood, pus or waste fluids from squirting into the Doctors face and body while the Doctor is performing any procedure that entails cutting into a human or animal. It is the correct scalpel to use when performing the initial incision or cut.

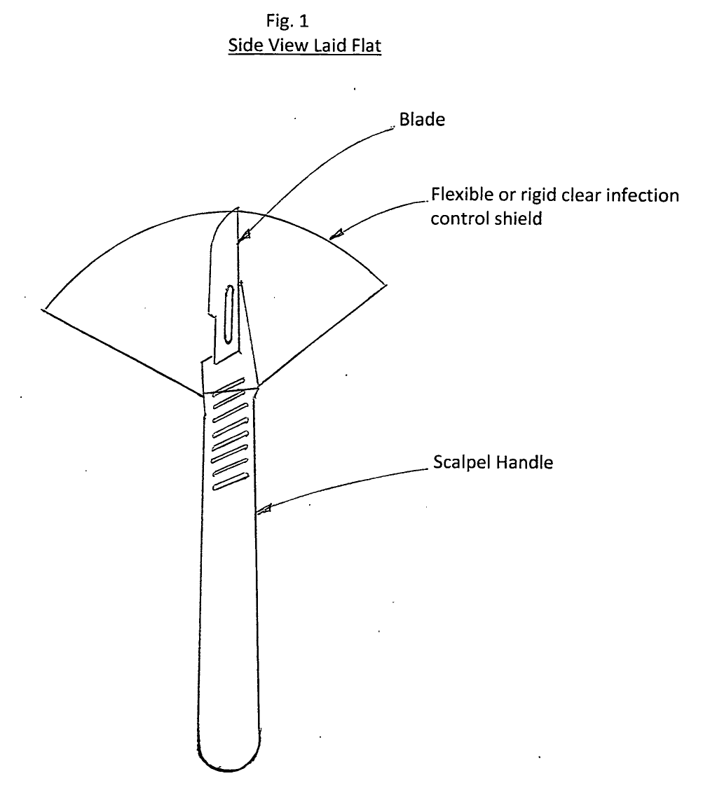

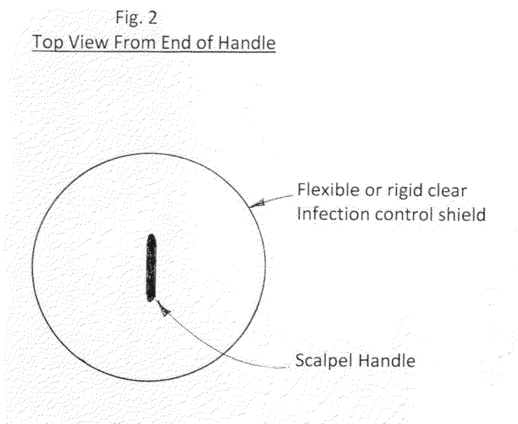

### BACKGROUND OF THE INVENTION

This invention pertains to the field of medical surgery and can be used on humans, animals and reptiles.

It was observed that during medical procedures involving the use of a scalpel for piercing and cutting skin lesions, abscesses and cysts that the patient's blood, pus or bodily fluids would often squirt onto the Doctors or Nurses face, body and the medical premises creating the very high possibility of infection in staff and clients. This type of event also necessitates the additional expense of cleaning and sanitizing the premises. The use of this Infection Control Scalpel will prevent the downtime necessary to call the relevant staff and have the premises cleaned and sanitized. The combined savings in Infection control and downtime loss will save each institution a significant amount of dollars. This invention modifies every kind of scalpel and cutting device by providing a clear shield that prevents blood or pus from flying in every direction.

### BRIEF SUMMARY OF THE INVENTION

This invention pertains to the field of medical surgery and can be used in humans, animals and reptiles. The invention is a modified scalpel or cutting tool with a skirt attached around the shaft and blade that captures and controls bodily fluids from spreading onto medical staff and future users of the room or surgical facility, and thus controlling the spread of infection.

It was observed that during medical procedures involving the use of a scalpel for piercing and cutting skin lesions, abscesses and cysts that the patient's blood, pus or other bodily fluids or waste, would often squirt onto the Doctors or Nurses face, body and the medical premises creating the very high possibility of infection in staff and clients. This type of event also necessitates the additional expense of cleaning and sanitizing the premises. The use of this scalpel will prevent the downtime necessary to call the relevant staff and have the premises cleaned and sanitized. The combined savings in Infection control and downtime loss will save each institution a significant amount of dollars.

The objective was to modify existing scalpels and cutting devices by providing a clear shield that prevents blood or pus from flying in every direction, without obstructing the Doctors or Nurses vision to perform such procedures.

### DETAILED DESCRIPTION OF THE INVENTION

This invention pertains to the field of medical surgery and can be used on humans, animals and reptiles.

The invention is a scalpel or cutting device that has a clear flexible or rigid shield built or attached above the blade and on the shaft, that is clear and does not obstruct the users vision during the performance of any procedure. The base scalpel or cutting device may be made of a variety of materials and come in various configurations and sizes. The blade may be retracting, flexible or rigid.

The scalpels and cutting devices available in the market currently do not have any such material to prevent the squirting of blood or pus from flying in every direction and causing serious infection consequences, besides the additional cost of closing the infected area temporarily and bringing in janitorial staff to clean and disinfect the affected area. The Infection Control Scalpel captures these materials within its folds and lets them drip down harmlessly on or about the patient. In the last few years several scalpel modifications have been made and several patents have been granted by the US Patent Office. Most patents in process or granted have dealt with the blade, the placement of the blade, the safety of the blade, slide-able shields, covers and blade guards for scalpel blades.

In this invention a skirt is permanently attached to the scalpel or knife body at the factory. The skirt is attached above the blade to the shaft of the scalpel. Certain scalpels have movable blade covers that slide upward on, or towards, the shaft. In this case the skirt is attached to the moveable section of the blade shield or cover.

The skirt is made of vinyl, plastic or other clear material. The skirt may or may not have perforations made in the material, in the event the doctor or nurse wants to remove it at any time.

The invention claimed is:

1. A surgical scalpel that has a skirt preventing blood, pus or waste fluids from squirting in every direction while the Doctor is performing any procedure that entails cutting into a human or animal. This is the correct scalpel to use when performing the initial incision or cut. The skirt attached to the shaft of the knife or scalpel controls the dispersion of the body fluid and restricts its spray pattern to mostly within the confines of the skirt.

2. The invention is to be used on any cutting and piercing tool. The cutting blade may be made of any number of materials like steel, plastic, laser etc.
